(19) United States
(12) Patent Application Publication
(10) Pub. No.: US 2025/0198995 A1
(43) Pub. Date: Jun. 19, 2025

(54) Methods and Reagent Kits for Diagnosing Canine Pancreatitis by Utilizing Acylcarnitines as Biomarkers

(71) Applicant: AXR Pharma Inc., Taipei City (TW)

(72) Inventors: Ching-Fen Wu, Taipei City (TW); Yun-Lian Lin, Taipei City (TW); Yi-Yueh Wang, Taipei City (TW); Hung-Chih Kuo, Taipei City (TW); Kun-Wei Chan, Taipei City (TW)

(21) Appl. No.: 18/984,972

(22) Filed: Dec. 17, 2024

(30) Foreign Application Priority Data
Dec. 18, 2023 (TW) 112149334

(51) Int. Cl. G01N 33/53

(52) U.S. Cl. CPC G01N 33/5308; G01N 2800/067

(57) ABSTRACT

The present invention provides a method for diagnosing pancreatitis in a canine. The method involves the following steps: First, a specimen is obtained from a canine animal. Then, a concentration of a specific biomarker in the specimen is detected, and the detected concentration is compared with a predetermined threshold value. If the biomarker concentration exceeds the threshold, the canine is determined to have an increased risk of pancreatitis. The predetermined threshold is based on the biomarker concentration observed in healthy canine animals. The biomarker can include medium-chain acylcarnitine, long-chain acylcarnitine, or a combination of both. Medium-chain acylcarnitine refers to those molecules with 6-12 carbon atoms in the acyl chain. Long-chain acylcarnitine refers to those molecules with more than 12 carbon atoms in the acyl chain. Additionally, the invention provides a method for using acylcarnitines (medium-chain and/or long-chain) as biomarkers for diagnosing pancreatitis in canines.

### BACKGROUND OF THE PRESENT INVENTION

### Field of Invention

The present invention pertains to a method and reagent kit for diagnosing pancreatitis in canines. Specifically, it involves the use of specific biomarkers to detect diagnose pancreatitis in canine animals.

### Description of Related Arts

Acute pancreatitis occurs when trypsinogen is abnormally activated within the pancreas, causing trypsin inhibition of pancreatic cells, leading to self-digestion and inflammation. It is one of the most serious digestive tract diseases in dogs. Pancreatitis usually involves mild to moderate anorexia, abdominal pain, and vomiting. Appropriate symptomatic treatment yields complete recovery. However, severe case of acute pancreatitis, which are associated with extensive pancreatic necrosis, usually accompanies severe systemic inflammatory response and multiple organ dysfunction. In the severe cases, the mortality rate can approach 50%.

Currently, diagnosing acute pancreatitis in dogs remains challenging, particularly in assessing its severity. Serum canine pancreatic-specific lipase (cPL) is commonly used as a diagnostic marker, but its sensitivity and specificity still require improvement. Additionally, cPL cannot reliably distinguish the severity of acute pancreatitis. In clinical practice, accurate diagnosis depends on a combination of factors, including the animal's medical history, clinical symptoms, and abdominal ultrasound findings.

### SUMMARY OF THE PRESENT INVENTION

This invention provides a method for diagnosing or detecting canine pancreatitis and a reagent kit for such detection. By measuring the concentration of special biomarkers in biological specimens, the method enables rapid identification of potential canine pancreatitis and can assist in assessing the severity of acute pancreatitis in dogs.

In one embodiment of this invention, a method for diagnosing canine pancreatitis is provided. First, a specimen is obtained from a canine. Next, the concentration of a specific biomarker in the specimen is measured. If the concentration is higher than a predetermined value, the canine is determined to have a high risk of pancreatitis. The biomarker includes medium-chain acylcarnitine, long-chain acylcarnitine, or a combination of both, where medium-chain refers to those with 6-12 carbon atoms, and long-chain acylcarnitine refers to those with more than 12 carbon atoms.

According to another embodiment of this invention, a diagnostic kit for detecting canine pancreatitis is provided. The kit includes a detection reagent that, when combined with a canine specimen, can measure—the concentration of specific biomarkers in the specimen. The biomarkers include medium-chain acylcarnitine, long-chain acylcarnitine, or their combinations. “Medium-chain” refers to acylcarnitines with 6 to 12 carbon atoms, while “long-chain” refers to those with more than 12 carbon atoms.

According to another embodiment of this invention, a method is provided for diagnosing canine pancreatitis by utilizing medium-chain acylcarnitine, long-chain acylcarnitine, or a combination of both. In this context, “medium-chain” refers to those acylcarnitines containing 6-12 carbon atoms, while “long-chain” refers to those with more than 12 carbon atoms.

In one embodiment of this invention, the aforementioned medium-chain acylcarnitine biomarkers include Decanoyl-L-carnitine (C10), Dodecanoyl-L-carnitine (C12), or Dodecenoyl-L-carnitine (C12:1). The specimen used for analysis may include urine or serum.

In another embodiment of this invention, the aforementioned long-chain carnitine biomarker refers to Tetradecadienoyl-L-carnitine (C14:2), and the specimen includes either urine or serum.

By detecting the special biomarkers described in this invention, namely medium-chain acylcarnitine, long-chain acylcarnitine, or their combination—this method can assist in diagnosing canine pancreatitis. Early detection of a high probability of pancreatitis allows for immediate treatment, which can significantly improve the survival rate of affected canine animals.

### BRIEF DESCRIPTION OF THE DRAWINGS

FIG. 1 is a bar graph illustrating the relative levels—of 15 different acylcarnitines with varying carbon lengths in the serum of healthy dogs and dogs diagnosed with pancreatitis.

FIG. 2 is a bar graph illustrating the relative levels of 13 different acylcarnitines with varying carbon lengths in the urine of healthy dogs and dogs diagnosed with pancreatitis.

### DETAILED DESCRIPTION OF THE INVENTION

The detailed description and technical contents of the present invention are described:

To facilitate a better understanding of the invention for those skilled in the art, the following description will provide exemplary, with reference and accompanying figures to explain in detail both the technical contents of the invention and the intended effects.

The exact causes of acute pancreatitis remain unclear, but several factors are commonly speculated to contribute, including: a high-fat diet, poor dietary habits, obesity, drugs and toxins, endocrine diseases, and genetic predispositions. In dogs, pancreatitis is most frequently observed in middle-aged and elderly dogs, with certain breeds being more susceptible, such as miniature schnauzers. Acylcarnitine is one of the intermediate products of fatty acid metabolism, produced by esterification of fatty acids and L-carnitine. Since fatty acid metabolism is tightly regulated, alterations in this process can influence acylcarnitine levels, making it a useful biomarker for fatty acid oxidation disorders and metabolism-related diseases. When the β-oxidation pathway of fatty acids is impaired, acylcarnitine levels will significantly increase. As a product of fatty acid metabolism, acylcarnitine is found in body fluids such as serum and urine. Moreover, acylcarnitine has been shown to activate inflammation-related signaling pathways, and at elevated concentrations, it can directly affect normal cellular physiological functions.

To address the lack of effective biomarkers for detecting canine pancreatitis in previous technologies—this invention provides a method for diagnosing canine pancreatitis. The method begins with obtaining—a specimen from a canine animal. In one embodiment of this invention, canine animals include, but are not limited to: dogs, wolves, jackals, or foxes. In a preferred embodiment, canine refers to dogs. On the other hand, specimens can be obtained using various methods that separate the sample from the canine animal to serve as an independent source for testing. In one embodiment, specimens can be various tissue fluids or body fluids. In a preferred embodiment, the specimen refers to blood, particularly serum, which can be obtained through methods such as blood collection. In another preferred embodiment, the specimen refers to urine, which can be obtained either through the collection voided urine, or via invasive methods such as catheterization or cystocentesis (bladder aspiration).

Then, the concentration of a biomarker in the specimen is measured. If the concentration is higher than a predetermined value, the canine is considered to have a high risk of pancreatitis. In one embodiment of this invention, the biomarker refers to medium-chain acylcarnitine, long-chain acylcarnitine, or a combination of both, where medium-chain refers to those containing 6 to 12 carbon atoms, and long-chain refers to those with more than 12 carbon atoms. In a preferred embodiment of this invention, the biomarker refers to medium-chain acylcarnitine. When both serum and urine specimens are tested simultaneously, the preferred biomarkers include Decanoyl-L-carnitine (C10), Dodecanoyl-L-carnitine (C12), or Dodecenoyl-L-carnitine (C12:1). In another most preferred embodiment, the biomarker refers to long-chain acylcarnitine, with Tetradecadienoyl-L-carnitine (C14:2) being the preferred biomarker when both serum and urine specimens are tested simultaneously.

The detection of biomarker concentrations, as aforementioned, utilizes liquid chromatography-mass spectrometry (LC/MS). In this case, if the biomarker concentration in a test specimen is higher than a predetermined value, the canine animal is considered to be at high risk of pancreatitis. The predetermined value is based on the concentration of biomarkers in healthy canine animals, which serves as a reference value for comparison. The predetermined value for each biomarker is obtained through the following method: First, a healthy canine animal of the same species as the test canine animal (e.g., a domestic dog) is selected. A biological specimen is then collected from this healthy animal, such as serum from blood or urine. Using LC/MS or other suitable instruments, the concentration of the target biomarker (e.g., dodecanoyl carnitine) is measured in the healthy specimen. Next, the biomarker concentration in the test specimen is compared to that in the healthy specimen. If the biomarker concentration in the test specimen exceeds the healthy by a defined factor, the test canine animal is considered to be at high risk for pancreatitis. For example, in one embodiment of this invention, if the biomarker concentration in the serum specimen of the test animal is more than twice that of the healthy specimen, the test canine animal is considered at high risk for pancreatitis. Similarly, if the specimen is urine and the biomarker concentration in urine specimen exceeds the healthy specimen by more than 20 times, the canine animal is considered to be at high risk of pancreatitis.

By comparing the measured value to a predetermined threshold, it can be determined whether the canine animal is at high risk for pancreatitis. In this context a “high risk of pancreatitis” means that the canine animal exhibits clinical symptoms and diagnostic findings consistent with pancreatitis, as determined through traditional diagnostic methods. These may include clinical symptoms such as vomiting, diarrhea, loss of appetite, abdominal pain (with at least two of these symptoms presented); canine-specific lipase (cPL) value: more than 200 g/L, or full abdominal ultrasound examination showing: hypoechoic pancreatic parenchyma, pancreatic thickening, blurred pancreatic margins, surrounding hyperechoic fat tissue, and other abnormalities such as dilation of the pancreatic duct/bile duct and ascites. Other diagnostic indicators may also be considered if they are sufficient to suggest the presence of pancreatitis.

In another embodiment, the invention provides a diagnostic kit for canine pancreatitis. The kit includes a detection reagent. When the detection reagent is mixed with a canine specimen, the concentration of specific biomarker was detected. The biomarkers refer to medium-chain acylcarnitine and long-chain acylcarnitine, or a combination of both, where medium-chain refers to those with 6 to 12 carbon atoms, and long-chain refers to those with more than 12 carbon atoms. The detection reagent contains internal standards necessary or accurately measuring the concentration of these acylcarnitines. The quantitative measurement of medium/long-chain acylcarnitine concentrations can be performed using various methods known in the field, such as liquid chromatography-mass spectrometry (LC-MS) or high-performance liquid chromatography (HPLC). In one embodiment, the kit described in this invention can utilize various diagnostic methods, including enzyme-linked immunosorbent assay (ELISA), affinity chromatography column purification, or modified tube binding analysis methods to non-invasively diagnose medium/long-chain acylcarnitine. For instance, ELISA method is employed to detect canine pancreatitis. In this process, the medium/long-chain acylcarnitine is first conjugated to a labeling substance, which may include but not limited to biotin, His tag, fluorescent substances (such as Cy3 or Cy5), or Digoxigenin (Dig). The concentration of medium/long-chain acylcarnitine in the sample is then quantified by measuring the intensity of absorbance or fluorescence. This analysis helps determine the potential risk of canine pancreatitis.

In yet another embodiment of this invention, a method is provided for diagnosing canine pancreatitis using medium-chain acylcarnitine, long-chain acylcarnitine, or a combination of both. Medium-chain refers to those containing 6 to 12 carbon atoms, while long-chain refers to those with more than 12 carbon atoms. The related method of implementation for this embodiment, including the use of biological specimens, and biomarkers, are consistent with those described in previous embodiments and will not be repeated here.

To more clearly describe the implementation methods and effects of this invention, the following examples and related experiments are presented with detailed steps.

The experimental process of this example is as follows:

### 1. This Research was Approved by the Institutional Animal Care and Use Committee of National Chiayi University, Taiwan (Approval No. 111026).

### 2. Case Collection

1) Inclusion criteria for diseased dogs
   Clinical symptoms: The dogs must exhibit at least two of following symptoms: vomiting, diarrhea, loss of appetite, abdominal pain.
   A. Canine pancreatic-specific lipase (cPL) value: The cPL levels of the dogs must be greater than 200 g/L.
   B. Full abdominal ultrasound examination: The ultrasound findings should include at least one of following symptoms: hypoechoic pancreatic parenchyma, pancreatic thickening, blurred pancreatic margins, surrounding hyperechoic fat tissue, and other abnormalities such as pancreatic duct/bile duct dilation and ascites
2) Exclusion criteria for sick dogs
   Dogs with any of the following characteristics are excluded:
      A. A history of primary gastrointestinal, hepatobiliary, and urinary diseases
      B. No detectable pancreatic abnormalities on ultrasound, or if the pancreas cannot be clearly visible on ultrasound.
      C. Clinical symptoms persisting for more than 7 days
3) Inclusion criteria for healthy dogs
   Dogs are considered healthy if they meet the following criteria:
      No clinical symptoms or physical findings
      f Normal results in blood tests, abdominal ultrasound, and cPL levels.

### 3. Blood and Urine Collection

1) Healthy dogs:
   Blood: Collected from the cephalic vein during health examination visits.
   Urine: Collected through voiding. Each sample is collected once.
2) Diseased dogs:
   Blood: Collected from the cephalic vein during veterinary clinic visits.
   Urine: Collected via voiding urination when possible. If voided urine collection is not feasible, cystocentesis is performed. Sedation (Alfaxalone, 0.3 mL/kg) is administered when necessary.

### 4. Sample Testing

Serum and urine metabolomics and lipidomics analysis were performed using an Agilent 1290 UHPLC coupled with a 6540-QTOF mass spectrometry (Agilent Technologies, Santa Clara, CA), following previously published methods [1]. To ensure analysis quality, blank samples and mixed quality control samples were included at the beginning of each batch and after every five samples to calibrate the analysis. Additionally, instrument performance was verified using 40 synthetic standards at the start of each analysis. Each sample was analyzed in triplicate, and total ion chromatograms were manually inspected for quality check. Data processing was carried out using-MZmine3 [2] and ADAP algorithm [3] for spectral peak detection. Peak identification was confirmed by matching against an in-house metabolite database [4]. Potential metabolite features were selected after matching and preprocessing steps to remove those with more than 50% missing values. The remaining data were input by replacing missing values with half of the minimum positive value. —The preprocessed data were then normalized by total peak areas, log-transformed, and auto-scaled before statistical analysis using MetaboAnalyst 5.0 [5]. The methods referenced in this paragraph can be found in the following literature:
[1] Liu C T, Raghu R, Lin S H, Wang S Y, Kuo C H, Tseng Y, Sheen L Y. Metabolomics of Ginger Essential Oil against Alcoholic Fatty Liver in Mice. J. Agric. Food Chem. 2013; 61:11231-11240.
[2] Schmid, R., Heuckeroth, S., Korf, A. et al. “Integrative analysis of multimodal mass spectrometry data in MZmine 3.” Nature Biotechnology (2023); https://mzmine.github.io/.
[3] https://mzmine.github.io/mzmine_documentation/module_docs/lc-ms_featdet/featdet_adap_chromatogram_builder/adap-chromatogram-builder.html
[4] In-house database: the National Taiwan University MetaCore Metabolomics Chemical Standard Library.
[5] https://www.metaboanalyst.ca/docs/About.xhtml

### Experiment One: Canine Serum Testing

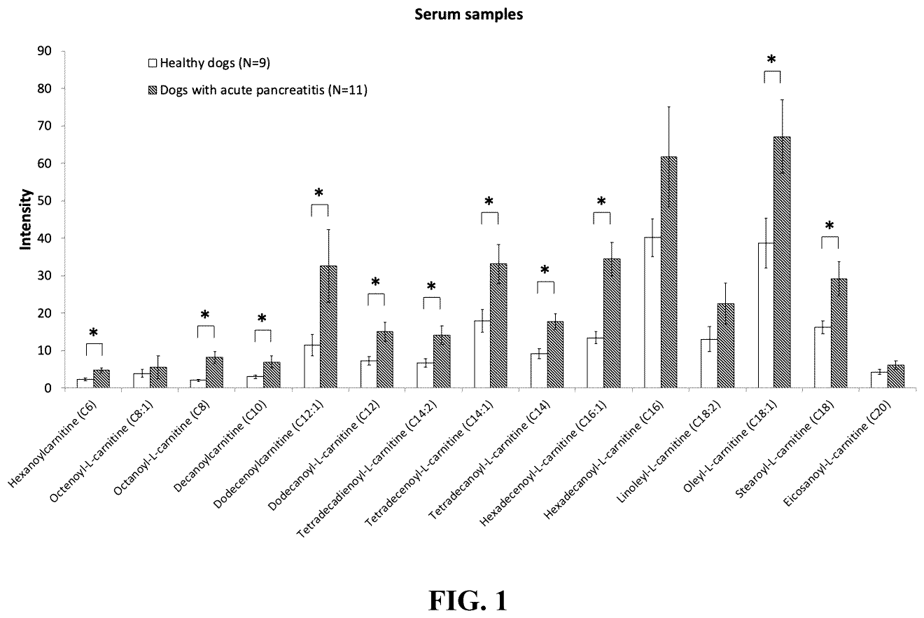

Serum from healthy dogs were compared to those dogs with pancreatitis, focusing on the levels of detected fifteen different acylcarnitines with varying carbon chain lengths. Please refer to FIG. 1, which shows bar graphs of the relative content of fifteen groups of acylcarnitines with different carbon chain lengths in serum from healthy dogs and dogs with pancreatitis. These acylcarnitines included: Hexanoyl L-carnitine (C6), Octenoyl-L-carnitine (C8:1), Octanoyl-L-carnitine (C8), Decanoyl-L-carnitine (C10), Dodecenoyl L-carnitine (C12:1), Dodecanoyl-L-carnitine (C12), Tetradecadienoyl-L-carnitine (C14:2), Tetradecenoyl-L-carnitine (C14:1), Tetradecanoyl-L-carnitine (C14), Hexadecenoyl-L-carnitine (C16:1), Hexadecanoyl-L-carnitine (C16), Linoleyl-L-carnitine (C18:2), Oleyl-L-carnitine (C18:1), Stearoyl-L-carnitine (C18), Eicosanoyl-L-carnitine (C20). As shown in FIG. 1, compared to healthy dogs, serum acylcarnitine levels were significantly higher in dogs with pancreatitis. Specifically, the levels of all acylcarnitine groups were increased in the pancreatitis group, with the following fold changes: C6 (2.04×), C8:1 (1.42×), C8 (3.89×), C10 (2.29×), C12:1 (2.85×), C12 (2.07×), C14:2 (2.11×), C14:1 (1.83×), C14 (1.94×), C16:1 (2.57×), C16 (1.53×), C18:2 (1.72×), C18:1 (1.73×), C18 (1.79×), and C20 (1.42×). These findings suggest that both medium-chain (6-12 carbon atoms) and long-chain (more than 12 carbon atoms) acylcarnitines are significantly elevated in dogs with pancreatitis, indicating their potential as biomarkers for this condition.

### Experiment Two: Canine Urine Testing

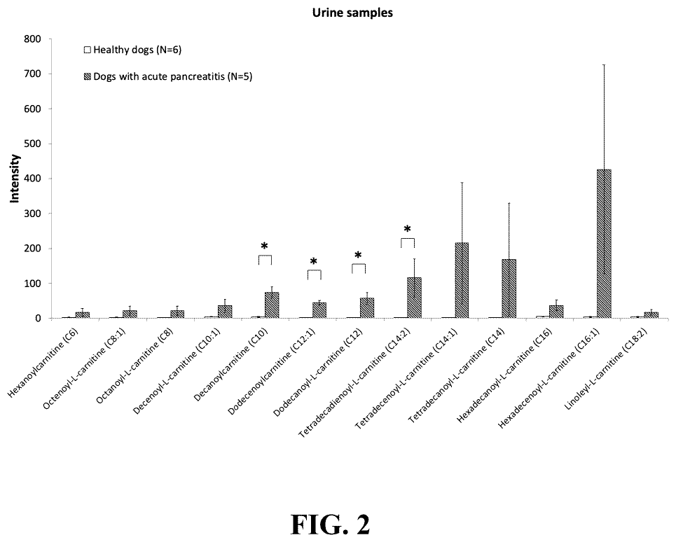

A comparison was made between urine samples from healthy dogs and those with pancreatitis, focusing on the detection—of thirteen groups of acylcarnitines with different carbon chain lengths. These groups, which are depicted in FIG. 2, shows bar graphs of thirteen groups of acylcarnitines with different carbon chain lengths in urine from healthy dogs and dogs with pancreatitis. The thirteen groups include: Hexanoyl L-carnitine (C6), Octenoyl-L-carnitine (C8:1), Octanoyl-L-carnitine (C8), Decenoyl-L-carnitine (C10:1), Decanoyl-L-carnitine (C10), Dodecenoyl-carnitine (C12:1), Dodecanoyl-L-carnitine (C12), Tetradecadienoyl-L-carnitine (C14:2), Tetradecenoyl-L-carnitine (C14:1), Tetradecanoyl-L-carnitine (C14), Hexadecanoyl-L-carnitine (C16), Hexadecenoyl-L-carnitine (C16:1), Linoleyl-L-carnitine (C18:2). As shown in FIG. 2, compared to healthy dogs, dogs with pancreatitis showed an increasing trend in acylcarnitines in their urine, with C10, C12:1, C12, C14:2 groups showing significant increases (p<0.05). The specific data for each group is presented, with the following fold changes, as follows: C6 (6.08×), C8:1 (9.40×), C8 (14.19×), C10:1 (8.28×), C10 (23.35×), C12:1 (24.38×), C12 (23.61), C14:2 (75.23×), C14:1 (120.14×), C14 (2086.68×), C16 (7.30×), C16:1 (114.01×), C18:2 (4.08×). This analysis indicates that both medium-chain and long-chain acylcarnitines in the urine of dogs with pancreatitis are substantially elevated compared to healthy dogs, which can effectively distinguish between healthy dogs and dogs with pancreatitis. The concentration of these acylcarnitines is, on average, at least 20 times higher in dogs with pancreatitis, supporting their potential as reliable biomarkers for this condition.

Based on the results from above Experiments One and Two, it is evident that medium-chain acylcarnitines show significant differences in serum and urine concentrations between healthy dogs and dogs with pancreatitis. Specifically, Decanoyl-L-carnitine (C10), Dodecenoyl-L-carnitine (C12:1), and Dodecanoyl-L-carnitine (C12) can be used as indicators for diagnosing canine pancreatitis. These compounds can be measured in both serum and urine, making them suitable for diagnostic testing. Additionally, among the long-chain acylcarnitines, Tetradecadienoyl-L-carnitine (C14:2) also prove to be a promising-screening marker for both urine and serum. Furthermore, urine sampling, being less invasive and easier to collect compared to serum, offers a more convenient and accessible option for home-based testing. This makes the reagent kit described in this invention more practical for use outside of clinical settings, eliminating the need for serum sampling, which is typically required for measuring cPL in existing diagnostic technologies. Consequently, the proposed method offers a simpler, more user-friendly alternative for early diagnosis of pancreatitis at home.

This invention provides specifically biomarkers, namely medium-chain acylcarnitine, long-chain acylcarnitine, or their combination, that can accurately assess the likelihood of pancreatitis in canine animals, such as dogs.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method for diagnosing pancreatitis in a canine, comprising:
obtaining a specimen from the canine;
measuring a concentration of a biomarker in said specimen, wherein the biomarker is selected from a group consisting of medium-chain acylcarnitine, long-chain acylcarnitine, and a combination thereof, wherein the medium-chain acylcarnitine refers to acylcarnitines with a chain length of 6 to 12 carbon atoms, and the long-chain acylcarnitine refers to acylcarnitines with a chain length of more than 12 carbon atoms;
comparing the measured concentration of the biomarker to a predetermined threshold value, wherein the threshold value is based on the concentration of the biomarker in a healthy canine; and
determining the risk of pancreatitis in the canine based on the comparison, wherein if the measured concentration exceeds the predetermined threshold value, the canine is determined to have a high risk of pancreatitis.

2. The method according to claim 1, wherein said medium-chain carnitine biomarker refers to Decanoyl-L-carnitine (C10), Dodecanoyl-L-carnitine (C12), or Dodecenoyl-L-carnitine (C12:1), and said specimen includes urine or serum.

3. The method according to claim 1, wherein said long-chain carnitine biomarker refers to Tetradecadienoyl-L-carnitine (C14:2), and said specimen includes urine or serum.

4. A diagnostic kit for pancreatitis in canine animals, comprising a detection reagent, wherein said detection reagent, when mixed with a specimen obtained from a canine animal, a concentration of a biomarker in the specimen can be detected, wherein the biomarker refers to medium-chain acylcarnitine, long-chain acylcarnitine, or a combination thereof, and the medium-chain refers to acylcarnitines with a chain length of 6 to 12 carbon atoms, and long-chain refers to acylcarnitines with a chain length of more than 12 carbon atoms.

5. The kit according to claim 4, wherein said medium-chain carnitine biomarker refers to Decanoyl-L-carnitine (C10), Dodecanoyl-L-carnitine (C12), Dodecenoyl-L-carnitine (C12:1), and said specimen includes urine or serum.

6. The kit according to claim 4, wherein said long-chain carnitine biomarker refers to Tetradecadienoyl-L-carnitine (C14:2), and said specimen includes urine or serum.

7. A method of utilizing medium-chain acylcarnitine, long-chain acylcarnitine, or a combination thereof for diagnosing pancreatitis in a canine, wherein medium-chain refers to acylcarnitines with a chain length of 6 to 12 carbon atoms, and long-chain refers to those with a chain length of more than 12 carbon atoms.

8. The method according to claim 7, wherein said medium-chain acylcarnitine refers to Decanoyl-L-carnitine (C10), Dodecanoyl-L-carnitine (C12), or Dodecenoyl-L-carnitine (C12:1), and said diagnosis uses urine or serum from the canine animal as the specimen.

9. The method according to claim 7, wherein said long-chain acylcarnitine refers to Tetradecadienoyl-L-carnitine (C14:2), and said diagnosis uses urine or serum from the canine animal as the specimen.
